SEQUENCE LISTING (8 sequences)

SEQ ID NO: 1 - DNA, 21 bp, Artificial Sequence
  HSD17B3-specific forward primer
aaatgtgataaccaagactgc

SEQ ID NO: 2 - DNA, 21 bp, Artificial Sequence
  HSD17B3-specific reverse primer
cttggtgttgagcttcaggta

SEQ ID NO: 3 - DNA, 23 bp, Artificial Sequence
  HSD17B12-specific forward primer
tgaatacttttggatgttcctga

SEQ ID NO: 4 - DNA, 24 bp, Artificial Sequence
  HSD17B12-specific reverse primer
agtcttggttgcagaatagatggt

SEQ ID NO: 5 - DNA, 24 bp, Artificial Sequence
  HSD17B11-specific forward primer
tggatataaaatgaaagcgcaata

SEQ ID NO: 6 - DNA, 24 bp, Artificial Sequence
  HSD17B11-specific reverse primer
atcagcttttggctaaagaacaag

SEQ ID NO: 7 - DNA, 19 bp, Artificial Sequence
  F7-specific forward primer
tcctgttgttggtgaatgg

SEQ ID NO: 8 - DNA, 19 bp, Artificial Sequence
  F7-specific reverse primer
gtacgtgctggggatgatg

(19) United States
(12) Patent Application Publication
(10) Pub. No.: US 2006/0166221 A1
(43) Pub. Date: Jul. 27, 2006

(54) Methods of diagnosing essential thrombocythemia

(76) Inventors: Wadie F. Bahou, Setauket, NY (US); Dmitri V. Gnatenko, Port Jefferson, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/040,385

(22) Filed: Jan. 21, 2005

(51) Int. Cl. C12Q 1/68

(52) U.S. Cl. 435/6

(57) ABSTRACT

This invention relates to diagnosis of essential thrombocythemia. More specifically, this invention provides methods of diagnosing essential thrombocythemia by detecting a decrease in gene expression or protein levels of type 3 17β-hyrdroxysteroid dehydrogenase, and an increase in gene expression or protein levels of type 12 17β-hyrdroxysteroid dehydrogenases, in a test subject.

### STATEMENT REGARDING FEDERALLY SPONSORED RESEARCH OR DEVELOPMENT

The work described in this invention was supported in part from grants from the U.S. National Institutes of Health, NHLBI (HL04239, HL76457 and HL49141). The government may have rights in this invention.

### FIELD OF THE INVENTION

This invention relates to diagnosis of essential thrombocythemia. More specifically, this invention relates to diagnosis of essential thrombocythemia by detecting changes in the gene expression or protein levels of type 3 and/or type 12 17β-hyrdroxysteroid dehydrogenases in the platelets of a test subject.

### BACKGROUND OF THE INVENTION

Circulating blood platelets are anucleate, although they retain small amounts of megakaryocyte-derived mRNAs and a fully functional protein biosynthetic capacity². Essential thrombocythemia (ET) represents a myeloproliferative disorder subtype, characterized by increased proliferation of megakaryocytes, elevated numbers of circulating platelets, and considerable thrombohemorrhagic events, not infrequently neurological³. ET is seen with equal frequency in males and females, although an additional female incidence peak at age 30 may explain the apparent higher disease prevalence in females. The molecular basis of ET remains to be established, although historically it has been considered a “clonal” disorder⁴. Causative mutations have been identified in the thrombopoietin gene. However, these mutations appear to be uncommon and restricted to rare individuals with familial thrombocythemia⁵. Other than the exaggerated platelet volume evident in subsets of ET platelets, the cells remain morphologically indistinguishable from their normal counterparts. No functional or diagnostic test is currently available for ET, and it remains to be diagnosed by exclusion.

The use of gene expression profiling in molecular classification of human cancer is well-documented. The feasibility of platelet profiling using apheresis techniques has also been demonstrated^{2,6}. However, it remains to be established whether gene expression profiling can be applied to diagnosis of poorly-understood myeloproliferative disorders, such as ET.

17β-hyrdroxysteroid dehydrogenases (17β-HSD) function in the formation and inactivation of all active androgens and estrogens, with substrate interconversion regulated by the oxidative state of the NADP/NAD(P)H cofactors⁹. To date, gene products encoding twelve types of 17β-HSD enzymes have been described, although the type 6 and 9 genes have been only characterized in rodents¹⁴. The HSD type 3 enzyme is generally regarded as testes-specific, although rare SAGE tags have been identified in CGAP tissues including brain (7×10⁻⁶), skeletal muscle (9.3×10⁻⁶), and prostate (1.6×10⁻⁵)¹⁰. The 17β-HSD3 enzyme specifically mediates the catalytic interconversion involving 4-androstenedione and testosterone. Molecular defects of the HSD17B3 gene are causally implicated in male pseudohermaphroditism¹¹. While steroidogenic pathways are incompletely characterized in platelets, it has been demonstrated that megakaryocytes (Mk) express the glucocorticoid receptor, and that both Mk and platelets selectively express estrogen receptor (ER) β and androgen receptor mRNA and protein, to the exclusion of ER α or progesterone receptor¹². Furthermore, Mk express functional 3β-HSD, known to catalyze the essential step in the transformation of 5-pregnen-3β-ol and 5-androsten-3β-ol steroids into the corresponding Δ⁴-3-keto-steroids, i.e., progesterone as well as the precursors of all androgens, estrogens, glucocorticoids and mineralocorticoids. Indeed, Mk-derived estradiol triggers megakaryocyte proplatelet formation in vitro, an effect that is blocked by inhibition of 3β-HSD activity¹³.

The present invention provides for the first time evidence showing that distinct subtypes of the steroidogenic 17β-HSDs are functionally expressed in human blood platelets, and that the expression patterns of HSD17B3 and HSD17B12 are distinctly associated with ET manifest by quantitative and qualitative platelet defects. Therefore, the present invention has identified the first diagnostic molecular signature for ET.

### SUMMARY OF THE INVENTION

The present invention provides methods of diagnosing ET based on detecting the levels of gene expression or the levels of the proteins of type 3 and type 12 17β-hyrdroxysteroid dehydrogenases in a test subject. Test subjects include any individuals of any age, more preferably to individuals having an abnormally high platelet count.

For the purpose of detecting gene expression or protein levels of type 3 and type 12 17β-hyrdroxysteroid dehydrogenases, a blood sample is taken from a test subject. Although not absolutely necessary, it is preferred that platelets are isolated from the blood sample for use in an assay. More preferably, isolated platelets are lysed in order to obtain mRNAs or cell lysates for use in the assay.

In one embodiment, diagnosis of ET is based on detecting a lower level of expression of the HSD17B3 gene in a subject, as compared to a control level (i.e., a level of gene expression in normal individuals without ET). Preferably, diagnosis is based on a level of HSD17B3 expression that is lower than a control level by at least 2-3 fold; more preferably, at least 4-6 fold.

In another embodiment, diagnosis of ET is based on detecting a higher level of expression of the HSD17B12 gene in a subject, as compared to a control level. Preferably, diagnosis is based on a level of HSD17B12 expression that is higher than a control level by at least 4-5 fold; more preferably, by at least 10-12 fold, or even 20-30 fold.

In yet another embodiment, diagnosis of ET is based on detecting a lower level of expression of the HSD17B3 gene, as well as a higher level of expression of the HSD17B12 gene in a subject, as compared to control levels of expression of the respective genes.

In still another embodiment, diagnosis of ET is based on determining the ratio of HSD17B12 gene expression versus HSD17B3 gene expression, and comparing the ratio with that obtained from normal subjects. An increased ratio is indicative of ET in a test subject. Preferably, the diagnosis is based on an increase in the HSD17B12:HSD17B3 ratio by at least 20 fold; even more preferably at least 50 fold; and most preferably, 75 to 100 fold.

In another embodiment, diagnosis of ET is based on determining the log₂ ratio of HSD17B12 gene expression versus HSD17B3 gene expression. Generally speaking, a log₂ ratio greater than 1 is indicative of ET in the test subject. Preferably, diagnosis is based on a log₂ ratio greater than 2, even more preferably, greater than 4.

In a further aspect of the invention, diagnosis of ET is based on detecting a lower level of the 17β-HSD3 protein, and/or a higher level of the 17β-HSD12 protein in a test subject, as compared to control protein levels. Similarly, diagnosis of ET can be based on detecting an increase in the ratio of the level of 17β-HSD12 versus the level of 17β-HSD3 in a subject, as compared to a control ratio.

### BRIEF DESCRIPTION OF THE DRAWINGS

FIGS. 1A-1C. Molecular signature of normal and thrombocythemic platelets. Gene expression profiles from 14 experimental samples (eleven apheresis donors [5 normal, NL A-E; 6 patients with essential thrombocythemia (ET)], or 3 normal leukocyte [WBC A-C] donors) are displayed. 1A. Relationships between the experimental samples are displayed as dendrograms, in which the pattern and length of the branches depict sample cohort relatedness among the experimental groups. 1B. Unsupervised hierarchical clustering using the 2,906-gene set demonstrates the distinct variation in gene expression pattern among defined cohorts; each row represents a single gene. 1C. Functional clusters using Gene Ontology classifications are delineated for the differentially-expressed gene set (N=170) identified by one-way ANOVA (p<0.01).

FIGS. 2A-2C. Identification of most significant differentially-expressed ET genes. Gene selection was calculated for each gene by applying a 5-fold cut-off and a computed t-statistic, using the aggregate (N=2906) gene list (2A), or the list of platelet-restricted (N=126) genes (2B). 2C. The list of 40 genes (20 over-expressed, 20 under-expressed) demonstrating greatest differential expression by one-way ANOVA is presented, ranked by the ratio of the mean group normalized signals (ET:Normal); shaded column delineates genes with highest expression in ET, whereas unshaded column refers to genes with lowest expression. Genes are delineated by gene symbol, or if unassigned, are specified by GenBank ID in parentheses. Genes in bold are platelet-restricted; 2/12 genes in 2B (ATOX1, LAPTM4B) are not depicted because they are present in 2C.

FIGS. 3A-3D. Transcript analysis of platelet-expressed HSD17Bs. Normalized microarray values for individual patients (3A-3B) or the normalized aggregate means by group (3C) are shown. In 3A (HSD17B3) and 3B (HSD17B12), expression levels are log₂-transformed, such that negative numbers reflect decreased expression compared to the normalized mean of 14 chips (6 ET, 5 normal, 3 WBC); for individual patients, delineation of transcript expression using Affymetrix MAS 5.0 software is specified (P—present; M—marginal; A—absent). 3D. Quantitative RT-PCR was completed on the original cohort of 6 ET patients and a new cohort of 10 normal controls (5 males, 5 females) using HSD17B3-, HSD17B11-, HSD17B12-, or F7-specific oligonucleotide primers (platelet coagulation factor VII (F7) is primarily endocytosed from plasma with negligible to no platelet mRNA expression³¹, thereby establishing the lower limit of assay sensitivity [1.1×10⁻⁵±2.6×10⁻⁷ ng/ng actin, not shown]). *p≦0.03; **p≦0.001.

FIGS. 4A-4C. Functional and genetic assays of normal and ET platelets. 4A. qRT-PCR was completed on platelets isolated by routine phlebotomy (10 mL) from ET or normal controls, using HSD17B-specific primers as outlined in FIG. 3D. The (log₂) ratio of HSD17B12:HSD17B3 transcript expression is depicted for individual patients, categorized by platelet count (10⁹/L) at time of platelet isolation; Category I: 0-350; Category II: 351-675; Category III: 676-999; Category IV: >1,000 (refer to Table 1 for detailed patient characteristics). Note that three Category I ET patient had normalized platelet counts with treatment at the time of analysis, and retained an HSD17B12:HSD17B3 ratio predictive of the ET phenotype; the single patient with secondary thrombocytosis (RT) studied to date had a normal ratio. 4B-4C. Platelet lysates (corresponding to 1.5×10⁸ platelets/sample) isolated from 3 normal or 3 ET patients were used for determination of functional 17β-HSD3 activity at distinct time points as outlined (control is HSD buffer alone). In 4C, curves generated for 30-minute (●) or 120-minute (▴) 17-βHSD3 assays (inset) were used as standards for comparative determination of platelet 17β-HSD3 assays performed in parallel. Data are the mean±SEM from 6 determinations; note that 0.1 testis equivalent units correspond to 10% of the activity found in molar-equivalent rat testes.

### DETAILED DESCRIPTION OF THE INVENTION

The present inventors have discovered that distinct subtypes of steroidogenic 17β-hyrdroxysteroid dehydrogenases (17β-HSDs) are functionally present in human blood platelets, and that the expression patterns of HSD17B3 and HSD17B12 are distinctly associated with essential thrombocythemia (ET), a myeloproliferative disorder characterized by increased proliferation of megakaryocytes and elevated numbers of circulating platelets. More specifically, it has been determined that the gene (HSD17B3) encoding type 3 17β-hydroxysteroid dehydrogenase is selectively down-regulated in ET platelets, with an induction of gene expression for the type 12 enzyme. Accordingly, the present invention provides diagnostic assays for ET based on detecting the levels of gene expression or the levels of the proteins of type 3 and type 12 17β-hyrdroxysteroid dehydrogenases in a subject.

As used herein, the abbreviation “17β-HSD” represents the protein form of steroidogenic 17β-hyrdroxysteroid dehydrogenase, and “HSD17B” (in italics) represents the corresponding nucleic acid encoding the 17β-hyrdroxysteroid dehydrogenase.

The present methods for diagnosing ET can be applied to any individuals of any age, more preferably to individuals having an abnormally high platelet count. Normal platelet counts generally range from 150,000/microliter of blood to 350,000/microliter of blood.

According to the present invention, the diagnostic methods can be either nucleic acid-based assays or protein-based assays. That is, the methods can be based on detecting the level of expression of the relevant gene, or based on detecting the level of the expressed protein product, in a sample taken from a test subject containing platelet.

Typically, a blood sample is taken from a test subject. The amount of blood taken may vary depending upon the particular assay to be used. Generally speaking, for nucleic acid-based assays, about 0.1 ml to 15 ml, or preferably about 1 ml to 10 ml of blood is taken from a subject. For protein-based assays, about 1 ml to 20 ml, or preferably about 5-10 ml, of blood is taken from a subject.

Although whole blood samples can be used directly in the assays as described herein, preferably, platelets are isolated from whole blood samples for use in the assays. Isolation of platelets can be achieved by standard techniques known in the art, e.g., apheresis, gel filtration and leukocyte immunodepletion by CD45-coupled magnetic micrbeads. More preferably, isolated platelets are further processed, e.g., lysed, in order to obtain mRNAs or cell lysates for use in the assays. The levels of mRNA of a gene of interest can be determined by a variety of assays, such as Northern Blot, RT-PCR, among others. The levels of a protein of interest can also be determined by using antibodies specific for the protein in standard assays, such as Western Blot, ELISA, among others.

In one embodiment, the present invention provides a method of diagnosing ET in a subject by detecting a lower level of expression of the HSD17B3 gene in the subject, as compared to levels of expression of the gene in normal individuals.

Levels of expression of the HSD17B3 gene in normal individuals, i.e., individuals without ET, can be determined in assays run side-by-side with the assay on a test subject. Alternatively, gene expression levels in normal individuals can be predetermined or established. A single value, such as the median or mean expression level, can be determined from a group or population of normal individuals for use as control in the diagnosis.

As described hereinabove, the level of expression of a gene, which is reflected by the level of the relevant mRNA, can be determined by a variety of assays, including Northern Blot analysis, RT-PCR, and the like.

According to the present invention, the diagnosis of ET is based on a level of HSD17B3 expression that is lower than a control level, preferably by at least 2-3 fold. More preferably, the diagnosis is based on a level of HSD17B3 expression that is lower than a control level by at least 4-6 fold.

In another embodiment, the present invention provides a method of diagnosing ET in a subject by detecting a higher level of expression of the HSD17B12 gene in the subject, as compared to levels of expression of the gene in normal subjects.

According to the present invention, the diagnosis of ET is based on a level of HSD17B12 expression that is higher than a control level, preferably by at least 4-5 fold. More preferably, the diagnosis is based on a level of HSD17B12 expression that is higher than a control level by at least 10-12 fold, or even 20-30 fold.

In yet another embodiment, the present invention provides a method of diagnosing ET in a subject by detecting a lower level of expression of the HSD17B3 gene, as well as a higher level of expression of the HSD17B12 gene in the subject, as compared to levels of expression of the genes in normal subjects.

In still another embodiment, the present invention provides a method of diagnosing ET in a subject by detecting the levels of expression of the HSD17B3 gene and the HSD17B12 gene in the subject, determining the ratio of HSD17B12 gene expression versus HSD17B3 gene expression, and comparing the ratio with that obtained from normal subjects. An increased ratio is indicative of ET in the test subject.

According to the present invention, diagnosis of ET based on the ratio of HSD17B12: HSD17B3 gene expression is more sensitive than diagnosis based on the change of expression of either gene alone. Preferably, diagnosis of ET is based on an increase in the HSD17B12: HSD17B3 ratio by at least 20 fold; and more preferably, at least 50 fold; and even more preferably, at least 75 to 100 fold.

In another embodiment, diagnosis of ET is based on determining the log₂ ratio (i.e., the log₂ value of the ratio) of HSD17B12 gene expression versus HSD17B3 gene expression. Generally speaking, a log₂ ratio greater than 1 is indicative of ET in the test subject. Preferably, diagnosis is based on a log₂ ratio greater than 2, even more preferably, greater than 4.

In a further aspect, the present invention provides a method of diagnosing ET in a subject by detecting a lower level of the 17β-HSD3 protein in the subject, as compared to levels of the 17β-HSD3 protein in normal individuals. As described hereinabove, the level of a protein can be determined using an antibody specific for the protein by a variety of assays, including Western Blot analysis, ELISA, among others.

According to the present invention, the diagnosis of ET is based on a level of the 17β-HSD3 protein that is lower than a control level determined from normal individuals, preferably by at least 2-3 fold. More preferably, the diagnosis is based on a level of the 17β-HSD3 protein that is lower than a control level by at least 4-6 fold.

In another embodiment, the present invention provides a method of diagnosing ET in a subject by detecting a higher level of the 17β-HSD12 protein in the subject, as compared to levels of the 17β-HSD12 protein in normal subjects.

The diagnosis of ET is based on a level of the 17β-HSD12 protein that is higher than a control level, preferably by at least 4-5 fold. More preferably, the diagnosis is based on a level of the 17β-HSD12 protein that is higher than a control level by at least 10-12 fold, or even 20-30 fold.

In yet another embodiment, the present invention provides a method of diagnosing ET in a subject by detecting a lower level of the 17β-HSD3 protein, as well as a higher level of the 17β-HSD12 protein in the subject, as compared to levels of the proteins in normal subjects.

In still another embodiment, the present invention provides a method of diagnosing ET in a subject by detecting the levels of the 17β-HSD3 protein and the 17β-HSD12 in the subject, determining the ratio of the two proteins (17β-HSD12:17β-HSD3), and determining an increase of the ratio when comparing to normal subjects as indicative of ET.

Alternatively, diagnosis of ET in a subject is based on determining the Log₂ ratio of the two proteins (17β-HSD12:17β-HSD3). A Log₂ ratio greater than 1 is indicative of ET in the test subject. Preferably, diagnosis of ET is based on a Log₂ ratio greater than 2; more preferably, by a ratio greater than 4.

The present invention is further illustrated by the following non-limiting example.

### EXAMPLE 1

Patient Selection and Characterization

Patients were enrolled from the larger pool of patients referred to the Division of Hematology for evaluation of thrombocytosis. All patients provided informed consent for an IRB (Institutional Review Board)-approved protocol completed in conjunction with the Stony Brook University Hospital General Clinical Research Center. Standard hematological criteria were followed for the diagnosis of essential thrombocythemia, reactive thrombocytosis, and other myeloproliferative disorders^{24,25}. Both sex- and age-distribution paralleled prevalence figures for ET, with a M:F ratio of 1:2.3, and age at diagnosis ranging from 23 to 78 years old. Platelet counts at the time of blood isolation ranged from normal (reflecting treatment) to 1,724,000/μL. Patient utilization of platelet-lowering drugs (i.e., hydroxyurea, analgrelide, or untreated) was recorded at the time of platelet isolation and purification. Detailed patient characteristics are set forth in Table 1.

Platelet Molecular Studies

Platelets were obtained by apheresis or from peripheral blood (10 mL), and were isolated essentially as previously described, utilizing gel-filtration and CD45-coupled magnetic micro-beads for leukocyte immunodepletion². The final platelet-enriched product contained no more than 3-5 leukocytes per 1×10⁵ platelets. Peripheral blood leukocytes from three healthy donors were isolated as previously described². Pure cellular pellets were resuspended in 10 mL of TRIZOL reagent (Invitrogen, Carlsbad, Calif.), transferred into DEPC (diethylpyrocarbonate)-treated Corex tubes, and serially purified and precipitated using isopropanol²⁶. Platelet mRNA quantification and integrity were established using an Agilent 2100 Bioanalyzer, and quantitative reverse transcription(RT)-PCR was performed using fluorescence-based real-time PCR technology (TaqMan Real-Time PCR, Applied Biosystems, Foster City, Calif.). Oligonucleotide primer pairs were generated using Primer3 software, designed to generate PCR products of approximately 200-bp at the same annealing temperature (71° C.). HSD17B3-specific primers were: forward (5′AAATGTGATAACCAAGACTGC 3′ [bp 755-775] (SEQ ID NO: 1)); reverse (5′CTTGGTGTTGAGCTTCAGGTA 3′ [bp 956-936] (SEQ ID NO: 2)); HSD17B12-specific primers were: forward (5′ TGAATACTTTTGGATGTTCCTGA 3′ [bp 496-519] (SEQ ID NO: 3)); reverse (5′ AGTCTTGGTTGCAGAATAGATGGT 3′ [bp 634-611] (SEQ ID NO: 4)); HSD17B11-specific primers were: forward (5′ TGGATATAAAATGAAAGCGCAATA 3′ [bp 1067-1090] (SEQ ID NO: 5)); reverse (5′ ATCAGCTTTTGGCTAAAGAACAAG 3′ [bp 1265-1242] (SEQ ID NO: 6)); F7-specific primers were: forward (5′ TCCTGTTGTTGGTGAATGG 3′ [bp 734-753] (SEQ ID NO: 7)); reverse (5′ GTACGTGCTGGGGATGATG 3′ [bp 933-915] (SEQ ID NO: 8)); β-actin-specific primers were as previously described². Purified platelet mRNA (4 μg) was used for first strand cDNA synthesis using random hexamers and SuperScript II reverse transcriptase (Invitrogen, Carlsbad, Calif.). For RT-PCR analysis, the RT reaction was equally divided among primer pairs and used in a 40-cycle PCR reaction for each target gene using two-step cycles of 94° C. for 20 seconds and 71° C. for 1 min. mRNA levels were quantified by monitoring real-time fluorimetric intensity of SYBR green I. Relative mRNA abundance was determined from triplicate assays performed in parallel for each primer pair, and calculated as previously described^{27,28}. For some patients, high molecular-weight genomic DNA was isolated from peripheral blood leukocytes for PCR-based amplification and sequencing of exon-intron boundaries²⁹.

Gene expression profiles were completed using the 22, 283 HU133A probe set (Affymetrix, Santa Clara, Calif.). Total cellular RNA (5.8 μg) was used for cDNA synthesis using SuperScript Choice system (Life Technologies, Rockville, Md.) and an oligo(dT) primer containing the T7 polymerase recognition sequence, followed by cDNA purification using phenol/chloroform extraction and ethanol precipitation. In vitro transcription was completed in the presence of biotinylated ribonucleotides using a BioArray HighYield RNA Transcript Labeling Kit (Enzo Diagnostics, Farmingdale, N.Y.). After metal-induced fragmentation, 10 μg of the biotinylated cRNA was hybridized to the oligonucleotide probe array for 16 hours at 45° C. After washing, the cRNA was detected with streptavidin-phycoerythrin (Molecular Probes, Eugene, Oreg.), and analysis was completed using a Hewlitt-Packard Gene Array Scanner. The fluorescence intensity of each probe was quantified using Affymetrix GeneChip software (MAS version 5.0), calculated as an average difference for each gene set obtained from 16 to 20 paired (perfectly matched and single nucleotide-mismatched) 25-bp oligonucleotides. The software was designed to exclude “positive calls” in the presence of high average differences with associated high mismatch intensities.

Bioinformatic and Statistical Analyses

Microarray data were visualized and analyzed using GeneSpring (version 7.0) software (Agilent, Palo Alto, Calif.). Data were normalized by dividing each measurement by the 50th percentile of all measurements in that sample, and each gene was divided by the median of its measurements in all samples. Normalized median ratios of individual genes were log₂-transformed, filtered for presence across arrays, and selected for expression levels as detailed. Prior to unsupervised hierarchical clustering of the uncentered Pearson correlation similarity matrix, the data were filtered for gene expression across phenotypic cohorts, defined as those genes present or marginal in a minimum of 80% of platelet samples (yielding 2,906 transcripts). A subset of genes was culled from the 2,906-gene list to specifically delineate those transcripts uniquely expressed in platelets; this platelet-restricted subset (N=126) was delineated by removing genes expressed in 3/3 leukocyte microarrays. A non-parametric analysis of variance test (ANOVA) was performed to identify differentially expressed genes using the Benjamini and Hochberg method to lower the false discovery rate (p<0.01). All statistical analyses were completed using SPSS (Statistical Package for Social Sciences, version 11.5) software.

Functional 17β-HSD Studies

Functional studies for platelet 17β-HSD3 activity were completed using gel-filtered platelets (GFP). Briefly, 1.5×10⁸ platelets were solubilized and freeze-thawed in HSD buffer containing (20 mM Tris-HCl (pH 8.0), 5 mM MgCl₂, 50 mM NaCl, 5% Glycerol, 10 mM DTT, 1.5 mM NAD (D-5755, Sigma Co., St. Louis, Mo.)). The reaction was started by addition of 1 μL (12.7 pM) of [1,2,6,7-³H(N)]-testosterone (specific activity 78.5 Ci/mM) (Dupont/NEN) to equivalent protein aliquots. A 30- or 90-minute reaction was allowed to proceed at 25° C., and quenched at −20° C. in the presence of cold testosterone (1 mg/mL) and androstenedione (1 mg/mL). Steroid extraction was performed twice using 150 mL of ethyl acetate. Both fractions were combined and air-dried under vacuum. Androstenedione and testosterone were then separated by thin layer chromatography using 4:1 (v/v) chloroform:ethyl acetate. Discrete steroid fractions were visualized and extracted from silica gels using ethanol, and quantified by liquid scintillography. Purified hydroxysteroid dehydrogenase from Pseudomonas testosteroni (Sigma Co., St. Louis, Mo.) diluted to 10 mg/mL in HSD buffer served as control for some experiments. Mouse testis extract prepared from a C57/B16 mouse served as standard for platelet 17β-HSD3 quantification, and was prepared by homogenization in 3 mL of HSD buffer. After centrifugation at 5000 g for 5 minutes, supernatants were gel-filtered in HSD-equilibrated Centrisep spin columns prior to use. Protein quantification of all samples was determined by Lowry method as previously described³⁰.

TABLE 1
Patient characteristics

| | | | | | Platelet count³ | | Genetic |
|---|---|---|---|---|---|---|---|
| ID | Age¹ | Sex | Diagnosis | Sample Source² | (×10⁹/L) | Treatment⁴ | analysis⁵ |
| ET1 | 31 | F | ET | B, P | 1,308 | H | M, P |
| ET3 | 49 | M | ET | B, P | 565 | H | M, P |
| ET4 | 33 | M | ET | B, P | 1,515 | N | M, P |
| ET5 | 23 | F | ET | B, P | 1,566 | N | M, P |
| ET6 | 37 | F | ET | B, P | 539 | A | M, P |
| ET7 | 65 | F | ET | B | 991 | A | P |
| ET9 | 64 | F | ET | B | 588 | A | P |
| ET10 | 57 | M | PV/ET⁶ | B | 945 | H | P |
| ET11 | 71 | F | ET | B | 477 | H | P |
| ET12 | 40 | F | ET | B | 456 | N | P |
| ET14 | 65 | F | ET | B | 853 | A | P |
| ET15 | 64 | F | ET | B | 511 | H | P |
| ET18 | 78 | M | ET | B | 329 (940) | H | P |
| ET19 | 77 | F | ET | B | 488 | H | P |
| ET20 | 69 | M | ET | B | 345 (1,063) | A | P |
| ET27 | 50 | F | PV/ET⁶ | B, P | 1,724 | H | M, P |
| ET28 | 29 | F | RT⁷ | B | 402 | N | P |
| ET29 | 66 | F | ET | B | 495 | N | P |
| ET31 | 70 | M | ET | B | 347 (880) | A | P |
| ET32 | 50 | F | ET | B | 513 | N | P |
| ET33 | 75 | F | ET | B | 641 | H | P |

¹Age at diagnosis
²Sample source: B - peripheral blood; P—plateletpheresis
³Platelet count at time of sample collection (normal range 150-350); note that for patients with normal platelet counts at time of blood isolation, the platelet counts in parentheses are highest pre-treatment determinations
⁴Refers to treatment at the time of platelet isolation; A—Anagrelide; H—Hydroxyurea; N—Not treated
⁵M—Microarray; P - Quantitative RT-PCR
⁶Originally given the diagnosis of polycythemia rubra vera (PV)
⁷Secondary (post-splenectomy) thrombocytosis

### EXAMPLE 2

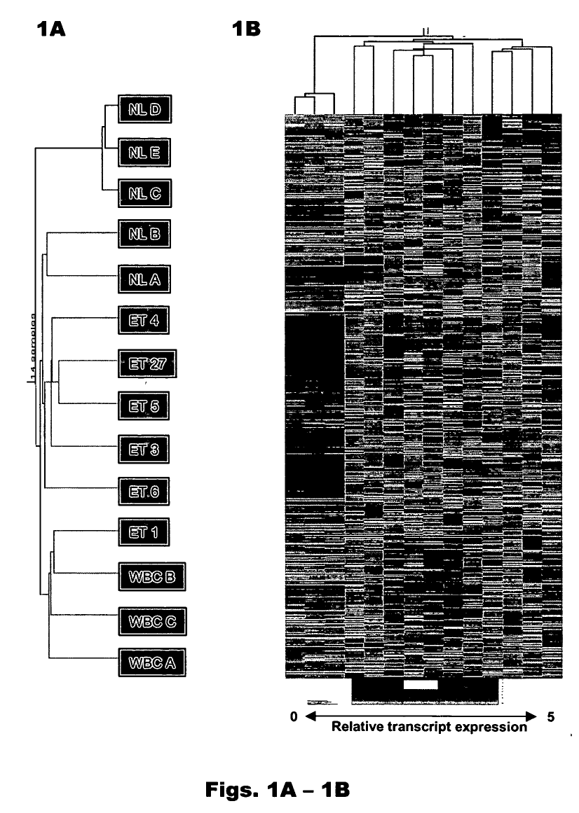

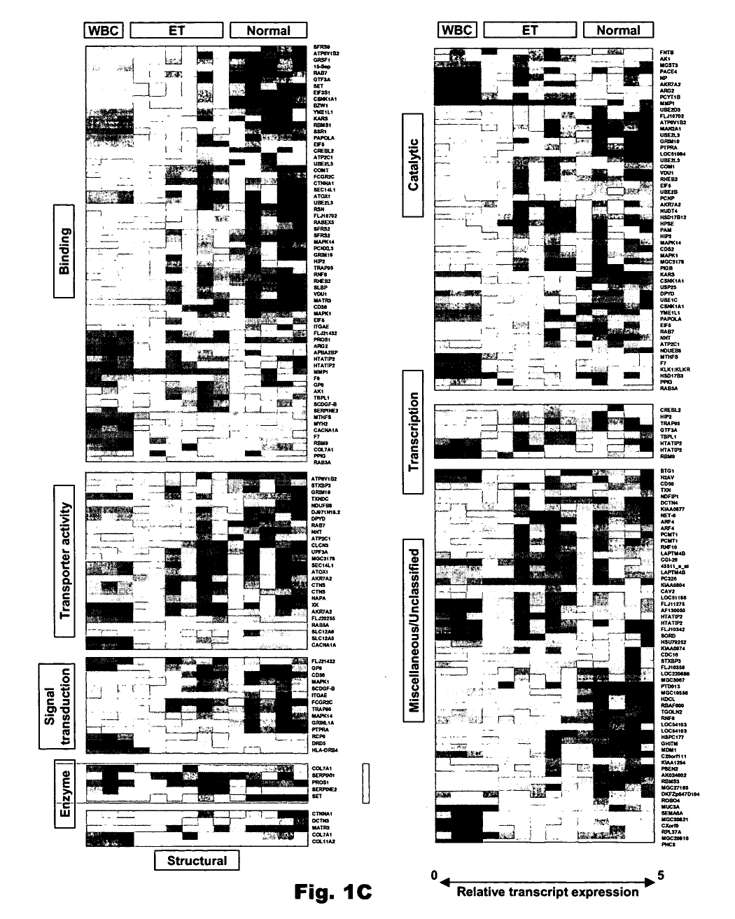

The genetic profiles of highly-purified apheresis platelets isolated from 6 ET patients (4 females, 2 males) and 5 normal, healthy controls demonstrated distinctly different molecular signatures (FIGS. 1A-1B). ET platelets collectively demonstrated higher numbers of expressed transcripts compared to normal controls, but considerably less than the transcript numbers generally found in nucleated cells². Of the genes classified as marginal or present in a minimum of 4 microarrays, ET patient samples expressed an average of 3,562 transcripts compared to 1,668 for normal controls (compared with approximately 10,530 transcripts identified in all three leukocyte microarrays). More stringent analyses (i.e. found in all of the arrays within a single group) extended these differences, with 1,840 transcripts expressed in ET platelets versus 1,086 transcripts expressed in platelets from healthy controls (p<0.03). Thus, while bone marrow megakaryocyte expansion is known to accompany the thrombocythemic phenotype, this cellular proliferation has now been shown to be also associated with a nearly 45% increase in overall gene transcription.

An unsupervised, hierarchical clustering algorithm was used to group normal and ET platelet genes on the basis of similarities of gene expression (FIG. 1B). This direct comparison against a genetically normal platelet pool highlighted genes that consistently distinguish normal from diseased platelets⁷. As can be seen from FIG. 1B, normal platelets and ET platelets had genetic profiles distinct from leukocytes. Additionally, differences between normal and ET platelets were also evident.

Several methods of computational analyses were completed to identify genes that could discriminate between ET and normal platelets. An initial one-way ANOVA analysis identified 170 genes that were differentially expressed, the majority of which (141) were up-regulated in ET platelets, with only 29 genes down-regulated in ET platelets compared to normal platelets. Functional cluster analysis of this limited set of differentially-expressed genes (FIG. 1C) demonstrated that genes involved in adhesion and catalytic activity represented the largest subgroups, although a sizable number of genes (31%) remained unclassified.

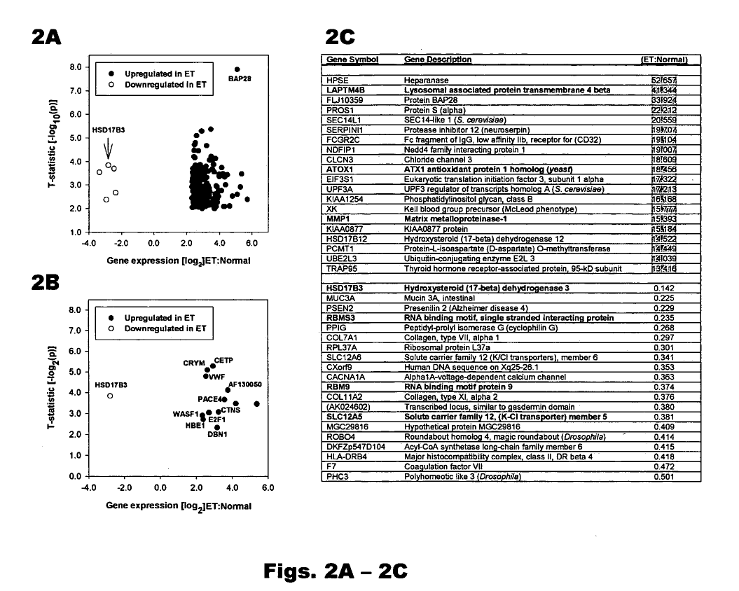

Because of inherent difficulties in analysis of microarray datasets, the data were re-analyzed by computing t-statistics of ET versus normal platelets for each gene. The t-values were ranked by absolute magnitude (thereby incorporating inter-sample variability in expression ratios), and were then ranked by the magnitude of the test statistic numerator, a measure of the biological difference in expression ratios. By applying a rigid 5-fold difference in pair-wise expression as the cut-off, 163 genes were identified that were up-regulated in ET. Only a small number (5) of genes were down-regulated (FIG. 2). To further pare this list, the analyses were repeated using the database of genes whose expression was restricted to platelets. This subset of platelet-restricted genes was delineated by excluding genes from the 2,906-gene list that were expressed in all 3 leukocyte array, leaving only 13 genes that were differentially-expressed (12 upregulated, 1 down-regulated in ET). Of the small subset of platelet-restricted, differentially expressed genes identified by this analysis, 8 were also in the top 40 list identified by the one-way ANOVA, establishing an independent layer of validity to these findings (FIG. 2C). While only 17% of the 170 differentially-expressed genes were underexpressed in ET platelets by one-way ANOVA, down-regulated genes were over-represented in the platelet-restricted gene list, accounting for 36% (9/25) of the total. Of the top 20 overexpressed genes, 3 encoded proteases or protease inhibitors (HPSE, MMPI, SERPINI1), a class of proteins well-characterized in tumor invasiveness and cancer metastases¹. Matrix metalloproteinases were recently been shown to mediate megakarycotye transendothelial migration and proplatelet formation, although this effect appeared restricted to MMP-9 to the exclusion of MMP-2⁸.

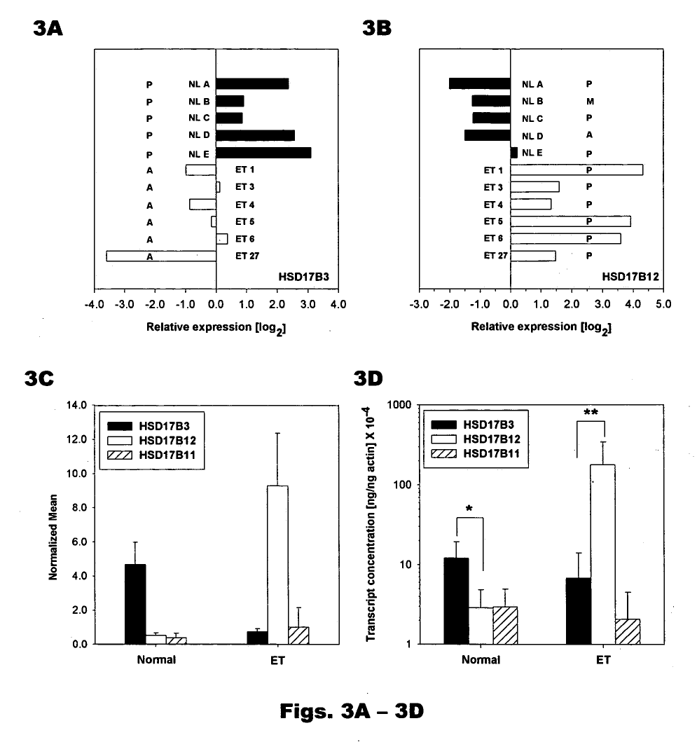

A single platelet-restricted gene HSD17B3 (encoding the type 3 17β-hyrdroxysteroid dehydrogenase [17β-HSD3]) was expressed in all normal arrays, and uniquely underexpressed in ET platelets compared to normal platelets (FIG. 2B). Review of the microarray data demonstrated that platelet HSD17B transcript expression was limited to three isoforms: HSD17B3, HSD17B11, and HSD17B12. While the HSD17B11 mean, normalized signals between the normal and diseased cohorts were low-level and not statistically different, there was a striking change in the pattern of HSD17B3 and HSD17B12 expression between ET and normal platelets (FIGS. 3A-3C). Loss of HSD17B3 transcript expression was evident in all 6 ET patients, changes that occurred concomitantly with elevated transcript levels of HSD17B12 in the same patient subgroup. In contrast, expression of HSD17B3 in normal platelets was accompanied by negligible to low-level HSD17B12 expression.

To validate and extend these results, a quantitative RT-PCR (qRT-PCR) assay was developed and was applied to the original ET cohort and an expanded cohort of normal controls, specifically collected to exclude potential gender-bias in HSD17B gene expression. The results paralleled those found by microarray, demonstrating approximately 4.5-fold greater HSD17B3 transcript (compared to HSD17B12) in normal platelets (p≦0.001) and concomitant approximately 27-fold greater HSD17B12 transcript expression (compared to HSD17B3) in ET platelets (p≦0.03). The reciprocal changes HSD17B3 and HSD17B12 amount to an aggregate about 2-log change in intracellular HSD17B3:HSD17B12 transcript levels between normal and ET platelets (FIG. 3D).

Because the qRT-PCR data established an absolute decrease in HSD17B3 transcript level in ET platelets, preliminary genomic analyses of HSD17B3 were completed in 4 of the 6 ET patients. The 11 exons and intron-exon boundaries were amplified and sequenced, with the identification of a single heterozygous A insertion (not involving the splice junction site¹⁵) in the first intron of one patient (ET1). Thus, there was no evidence that a small deletion or missense mutation affecting HSD17B3 transcript stability was causally implicated in the pathogenesis of HSD17B3 transcript loss.

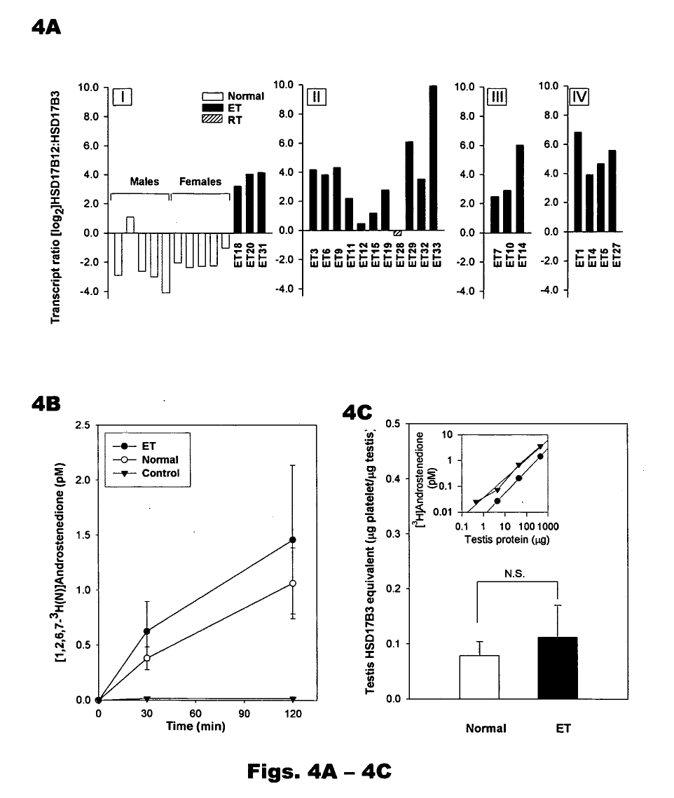

The distinct patterns of HSD17B expression identified by microarray and confirmed by qRT-PCR were then extended to a larger cohort of 20 ET patients (6 original ET patients and 14 newly-studied individuals), now uniformly analyzed using peripheral blood as the starting source for platelet analysis (unlike the cumbersome apheresis technique used in the original cohorts). The qRT-PCR results were entirely concordant for all individuals studied, demonstrating that 17BHSD12:17BHSD3 transcript ratios reliably predicted the ET phenotype in all patients studied to date (p<0.0001) (FIG. 4). Furthermore, these differential patterns of HSD17B expression appeared unrelated to the development of thrombocytosis per se, but rather, were restricted to the ET phenotype. This observation is based on the results of four individuals: ET28 who had secondary thrombocytosis and ratios predictive of the normal phenotype; and ET18, ET20, and ET31, who had aggressively-treated ET and normal platelet counts who maintain 17BHSD12:17BHSD3 transcript ratios predictive of ET. The causes of thrombocytosis are varied and require a study of larger cohorts of patients with etiologically diverse causes for thrombocytosis for confirmation. Nonetheless, the above data suggest that the intracellular signals regulating MK/platelet 17BHSD expression are associated with the molecular defect(s) causing ET, and not collectively applicable to the broader cohort of patients with secondary forms of thrombocytosis.

To confirm that platelets retained functional 17β-HSD3 activity (and to compare this activity between normal and ET platelets), the oxidative conversion of testosterone to 4-androstenedione was quantified. Entirely consistent with the genetic data, normal platelets retained 17β-HSD3 activity, providing for the first time evidence that non-testicular sources retain functional capacity in the penultimate step of androgen biosynthesis. Furthermore, the platelet-derived 17β-HSD3 activity was not inconsequential, providing nearly 10% of the capacity found in testis (FIGS. 4B and 4C). Finally, in the initial cohort of patients studied, ET platelets demonstrated a total 17β-HSD3 activity that was not statistically different from that found in normal platelets. Thus, the reciprocal induction of HSD17B12 transcript in ET platelets is not associated with overall enzymatic capabilities in androgen biosynthesis. These data suggest that the substrate specificity of 17β-HSD12 is distinct from that of 17β-HSD3.

### REFERENCES

1. Bahou, W. F. Protease-activated receptors. Curr Top Dev Biol 54, 343-369 (2003).
2. Gnatenko, D. V. et al. Transcript profiling of human platelets using microarray and serial analysis of gene expression. Blood 101, 2285-2293 (2003).
3. Nimer, S. D. Essential thrombocythemia: Another “heterogeneous disease” better understood? Blood 93, 415-416 (1999).
4. El-Kassar, N., Hetet, G., Briere, J. & Grandchanp, B. Clonality analysis of hematopoiesis in essential thrombocythemia: Advantages of studying T lymphocytes and platelets. Blood 89, 128 (1997).
5. Kondo, T. et al. Familial essential thrombocythemia associated with one-base deletion in the 5′-untranslated region of the thrombopoietin gene. Blood 92, 1091 (1998).
6. Bahou, W. F. & Gnatenko, D. V. Platelet transcriptome: the application of microarray analysis to platelets. Semin Thromb Hemost 30, 473-484 (2004).
7. Dhanasekaran, S. M. et al. Delineation of prognostic biomarkers in prostate cancer. Nature 412, 822-826 (2001).
8. Lane, W. J. et al. Stromal-derived factor 1-induced megakaryocyte migration and platelet production is dependent on matrix metalloproteinases. Blood 96, 4152-4159 (2000).
9. Labrie, F. et al. Endocrine and intracrine sources of androgens in women: inhibition of breast cancer and other roles of androgens and their precursor dehydroepiandrosterone. Endocr Rev 24, 152-182 (2003).
10. Strausberg, R. L., Buetow, K. H., Emmert-Buck, M. R. & Klausner, R. D. The cancer genome anatomy project: building an annotated gene index. Trends Genet 16, 103-106 (2000).
11. Geissler, W. M. et al. Male pseudohermaphroditism caused by mutations of testicular 17 beta-hydroxysteroid dehydrogenase 3. Nat Genet 7, 34-39 (1994).
12. Khetawat, G. et al. Human megakaryocytes and platelets contain the estrogen receptor beta and androgen receptor (AR): testosterone regulates AR expression. Blood 95, 2289-2296 (2000).
13. Nagata, Y. et al. Proplatelet formation of megakaryocytes is triggered by autocrine-synthesized estradiol. Genes Dev 17, 2864-2869 (2003).
14. Napoli, J. L. 17beta-Hydroxysteroid dehydrogenase type 9 and other short-chain dehydrogenases/reductases that catalyze retinoid, 17beta- and 3alpha-hydroxysteroid metabolism. Mol Cell Endocrinol 171, 103-109 (2001).
15. Mount, S. A catologue of splice junction sequences. Nucl. Acid Res. 10, 459-472 (1982).
16. Mindnich, R., Deluca, D. & Adamski, J. Identification and characterization of 17 beta-hydroxysteroid dehydrogenases in the zebrafish, Danio rerio. Mol Cell Endocrinol 215, 19-30 (2004).

17. Wattel, E. et al. Androgen therapy in myelodysplastic syndromes with thrombocytopenia: a report on 20 cases. Br J Haematol 87, 205-208 (1994).
18. Sullivan, P. S., Jackson, C. W. & McDonald, T. P. Castration decreases thrombocytopoiesis and testosterone restores platelet production in castrated BALB/c mice: evidence that testosterone acts on a bipotential hematopoietic precursor cell. J Lab Clin Med 125, 326-333 (1995).
19. Johnson, M., Ramey, E. & Ramwell, P. W. Sex and age differences in human platelet aggregation. Nature 253, 355-357 (1975).
20. Jones, S. B. et al. alpha 2-Adrenergic receptor binding in human platelets: alterations during the menstrual cycle. Clin Pharmacol Ther 34, 90-96 (1983).
21. Pilo, R., Aharony, D. & Raz, A. Testosterone potentiation of ionophore and ADP induced platelet aggregation: relationship to arachidonic acid metabolism. Thromb Haemost 46, 538-542 (1981).
22. Castellsague, J., Perez Gutthann, S. & Garcia Rodriguez, L. A. Recent epidemiological studies of the association between hormone replacement therapy and venous thromboembolism. A review. Drug Saf 18, 117-123 (1998).
23. Daly, E. et al. Risk of venous thromboembolism in users of hormone replacement therapy. Lancet 348, 977-980 (1996).
24. Iland, H. J. et al. Differentiation between essential thrombocythemia and polycythemia vera with marked thrombocytosis. Am J Hematol 25, 191-201 (1987).
25. Murphy, S., Peterson, P., Iland, H. & Laszlo, J. Experience of the Polycythemia Vera Study Group with essential thrombocythemia: a final report on diagnostic criteria, survival, and leukemic transition by treatment. Semin Hematol 34, 29-39 (1997).
26. Mirza, H., Yatsula, V. & Bahou, W. F. The proteinase activated receptor-2 (PAR-2) mediates mitogenic responses in human vascular endothelial cells. Molecular characterization and evidence for functional coupling to the thrombin receptor. J Clin Invest. 97, 1705-1714 (1996).
27. Heid, C. A., Stevens, J., Livak, K. J. & Williams, P. M. Real time quantitative PCR. Genome Res 6, 986-994 (1996).
28. Gnatenko, D. V. et al. Expression of therapeutic levels of factor VIII in hemophilia A mice using a novel adeno/adeno-associated hybrid virus. Thromb Haemost 92, 317-327 (2004).
29. Schmidt, V. A. et al. The human proteinase-activated receptor-3 (PAR-3) gene. Identification within a PAR gene cluster and characterization in vascular endothelial cells and platelets. J Biol Chem 273, 15061-15068 (1998).
30. Bahou, W. F., Scudder, L., Rubenstein, D. & Jesty, J. A shear-restricted pathway of platelet procoagulant activity is regulated by IQGAP1. J Biol Chem 279, 22571-22577 (2004).
31. Greenberg, D., Miao, C. H., Ho, W. T., Chung, D. W. & Davie, E. W. Liver-specific expression of the human factor VII gene. Proc Natl Acad Sci USA 92, 12347-12351 (1995).

What is claimed is:

1. A method of diagnosing essential thrombocythemia (ET) in a subject comprising obtaining a platelet-containing sample from said subject, and detecting the level of expression of the gene encoding type 3 17β-hyrdroxysteroid dehydrogenase (17β-HSD3) in said sample, wherein a lower level of expression when compared to a control level is indicative of ET in said subject.

2. The method of claim 1, wherein the level of expression of said gene coding for said 17β-HSD3 is lower than said control level by at least 3 fold.

3. A method of diagnosing ET in a subject comprising obtaining a platelet-containing sample from said subject, and detecting the level of expression of the gene encoding 17β-HSD12 in said sample, wherein a higher level of expression when compared to a control level is indicative of ET in said subject.

4. The method of claim 3, wherein the level of expression of said gene coding for said 17β-HSD12 is higher than said control level by at least 4 fold.

5. A method of diagnosing ET in a subject comprising obtaining a platelet-containing sample from said subject, detecting the levels of expression of the gene encoding 17β-HSD3 and the gene encoding 17β-HSD12 in said sample, wherein a lower level of gene expression of 17β-HSD3 in combination with a higher level of gene expression of 17β-HSD12, when compared to control levels, is indicative of ET in said subject.

6. A method of diagnosing ET in a subject comprising obtaining a platelet-containing sample from said subject, detecting the levels of expression of the gene encoding 17β-HSD3 and the gene encoding 17β-HSD12 in said sample, and determining the ratio of the level of gene expression of 17β-HSD12 relative to the level of gene expression of 17β-HSD3, wherein a higher value in the ratio when compared to a control ratio is indicative of ET in said subject.

7. The method of claim 6, wherein the ratio determined based on the sample from said test subject is higher than said control ratio by at least 20 fold.

8. A method of diagnosing ET in a subject comprising obtaining a platelet-containing sample from said subject, detecting the levels of expression of the gene encoding 17β-HSD3 and the gene encoding 17β-HSD12 in said sample, determining the Log₂ ratio of the level of gene expression of 17β-HSD12 relative to the level of gene expression of 17β-HSD3, and diagnosing ET based on said Log₂ ratio.

9. The method of claim 8, wherein said Log₂ ratio is greater than 1.

10. The method according to any one of claims 1, 3, 5-6 or 8, wherein said sample is a whole blood sample.

11. The method of claim 10, wherein said sample contains isolated platelets.

12. The method of claim 11, wherein said mRNAs are isolated from said platelets.

13. The method according to any one of claims 1, 3, 5-6 or 8, wherein the level of gene expression is determined based on the level of mRNA.
